(19) United States
(12) Patent Application Publication
(10) Pub. No.: US 2025/0228460 A1
(43) Pub. Date: Jul. 17, 2025

(54) Blood Pressure Monitoring

(71) Applicant: 1625986 Ontario Limited, Mt Brydges (CA)

(72) Inventors: Geoffrey Locke Taylor, Treasure Island, FL (US); Vitaliy Degtyary-Ov, Oakbank (CA)

(21) Appl. No.: 18/876,281

(22) PCT Filed: Jun. 27, 2023
(86) PCT No.: PCT/IB2023/056596

§ 371 Date: Dec. 18, 2024

Related U.S. Application Data
(60) Provisional application No. 63/356,089, filed on Jun. 28, 2022.

(51) Int. Cl. A61B 5/0205; A61B 5/021; A61B 5/024; A61B 5/0531; G16H 10/60

(52) U.S. Cl. CPC A61B 5/0205; A61B 5/0531; G16H 10/60; A61B 5/021; A61B 5/024; A61B 2560/0223

(57) ABSTRACT

The present invention provides for measurement of blood pressure using an interdigitated sensor. The measurements are based on capacitance, resistance, and phase characteristics of a subject's tissue.

### TECHNICAL FIELD

The present invention is in the technical field of noninvasive sensors responsive to blood pressure.

### BACKGROUND ART

High blood pressure (hypertension) can quietly damage the body for years before symptoms develop. Uncontrolled high blood pressure can lead to disability, a poor quality of life, or even a deadly heart attack or stroke. Treatment and lifestyle changes can help control high blood pressure to reduce the risk of life-threatening complications.

Low blood pressure can also relate to health problems. Studies have shown that people who had low diastolic blood pressure (60 to 69 mm Hg) were twice as likely to have subtle evidence of heart damage compared with people whose diastolic blood pressure was 80 to 89 mm Hg. Low diastolic values were also linked to a higher risk of heart disease and death from any cause. Aug. 30, 2016, Journal of the American College of Cardiology.

Conventional blood pressure measurements are based on use of an occlusive cuff. It consists of an inflatable cuff that is wrapped around your arm, roughly level with your heart, and a monitoring device that measures the cuff's pressure. The monitor measures two pressures: systolic, and diastolic. Systolic pressure is higher, occurring when your heart beats and pushes blood through the arteries, and diastolic pressure is measured when your heart is resting and filling with blood. So, for example, your blood pressure might be 120 over 80.

The cuff is placed around the arm, then inflated until it fits tightly around the arm, cutting off blood flow, and then the valve opens to deflate it. As the cuff reaches the systolic pressure, blood begins to flow around your artery. This creates a vibration that's detected by the meter, which records your systolic pressure. In a traditional analogue sphygmomanometer, the blood sounds are detected using a stethoscope. As the cuff continues to deflate, it reaches the diastolic pressure, and the vibration stops. The meter senses this, and records the pressure again.

Cuff-based measurements present difficulties. Measurements based on cuff techniques can be sensitive to arm position and other conditions. Resulting blood pressure measurements can demonstrate undesirable variability. Cuff-based measurements are not capable of measuring blood pressure continuously, which can be especially desirable when someone is doing something that can put their blood pressure in a dangerous range (low or high). Cuff-based measurements also require the blood flow be temporarily stopped, which is undesirable in some applications. The time required to complete a cuff-based measurement can complicate medical procedures or interventions responsive to blood pressure. Instantaneous or continuous blood pressure measurement can also be useful to monitor immediate influence of changes such as biological or emotional state.

### DESCRIPTION OF INVENTION

Embodiments of the present invention provide a device comprising (i) an interdigitated portion having two opposing ends and configured for contacting skin of a subject and measuring skin capacitance, resistance and phase; (ii) a capacitance meter; (iii) a resistance meter; and (iv) a phase meter responsive to the relationship between resistance and capacitance (also referred to as the relativity); and (v) an indication system that provides a signal representative of the difference of the current skin capacitance, resistance and phase measurements from a comparative capacitance, resistance, and phase value determined at a known blood pressure at a different time. The signal can be continuous if the measurements are continuous, can be at fixed periods, can be at the initiation of user, or can be triggered by an activity or condition (e.g., increased heart rate). The capacitance meter and resistance meter can be provided as separate meters, or can be provided in a single sensor system. The signal can indicate a change in blood pressure from detection of a change in capacitance, resistance, or phase. The signal can also indicate a trend or direction of change (e.g., increasing or decreasing, or rate of increase or decrease), or a value of systolic, diastolic, or both, blood pressure if the capacitance, resistance, and phase is calibrated, e.g., by corelating measurements with known blood pressures in this user or in a representative population.

Embodiments of the present invention provide a method for non-invasively detecting a subject's pulse and blood pressure, comprising providing a device comprising a number of interdigitated portions for measuring capacitance, resistance and phase of surface and the various layers below the surface of the skin of the subject, said interdigitated portion having at least two separate electrical contacts and at least two different spacings; a power source; and a signal responsive to one end of the interdigitated portion, said signal being activated when a skin capacitance, resistance and phase measurement of the subject changes compared to values of the subject when a blood pressure reading was previously taken; placing the device on a body of the subject such that two electrical contacts of the interdigitated portions are contacting the skin of the subject; said device measuring the capacitance, resistance and phase of the skin of the subject; and said device activating the signal when the skin capacitance resistance and phase measurement is different from the reference capacitance, resistance and phase measurement of the subject.

### BRIEF DESCRIPTION OF THE DRAWINGS

FIG. 1 is a schematic diagram of a circuit diagram of a capacitance meter of the present invention.

FIG. 2 is a schematic diagram showing three differently spaced interdigitated contact sensors.

FIG. 3 is an illustration of blood pressure and resistance.

FIG. 4 is an illustration of blood pressure and capacitance.

FIG. 5 is an illustration of blood pressure and phase.

FIG. 6 is an illustration of blood pressure and resistance.

FIG. 7 is an illustration of blood pressure and phase.

FIG. 8 is a schematic illustration of varying systolic and diastolic blood pressure.

FIG. 9 is a schematic illustration of an example embodiment.

### MODES FOR CARRYING OUT THE INVENTION AND INDUSTRIAL APPLICABILITY

### Example Embodiment

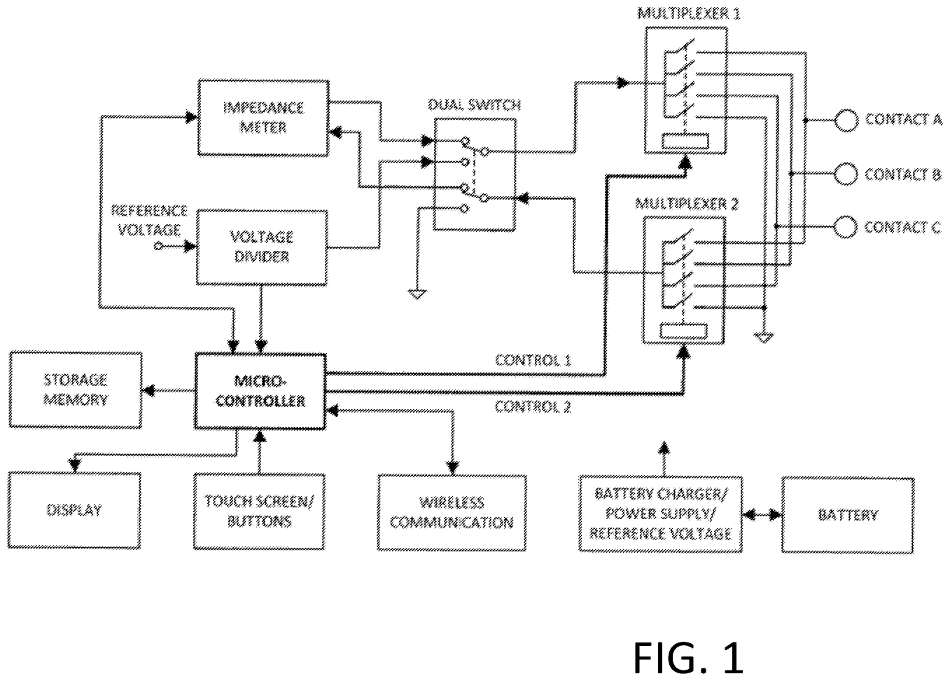

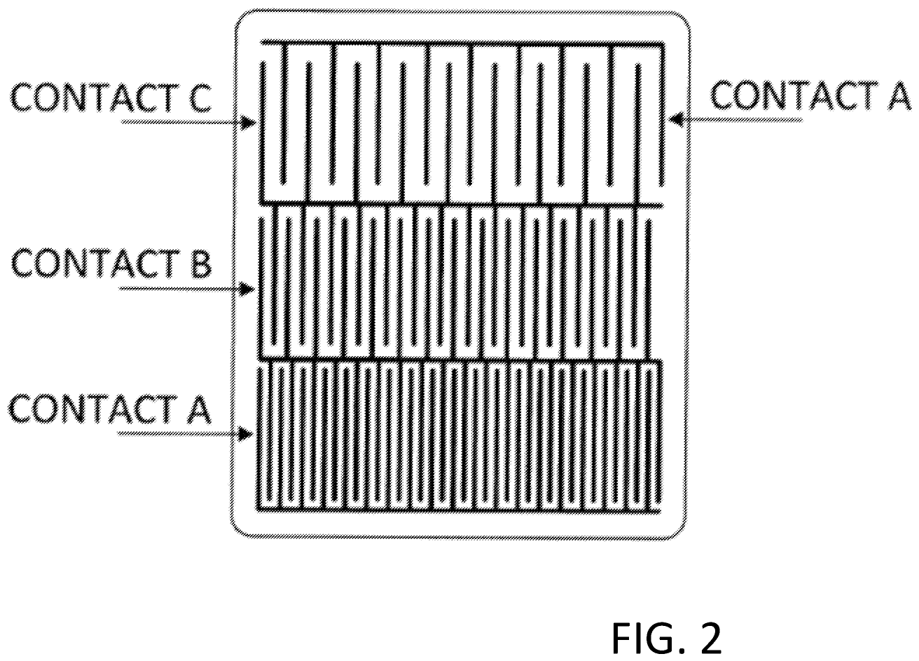

FIG. 1 is a schematic illustration of an example apparatus according to the present invention. The apparatus comprises an impedance meter and a voltage divider, connected through a dual switch to multiplexers and connect corresponding signals to an interdigitated sensor as in FIG. 2. A microcontroller receives and transmits signals to the impedance meter, voltage divider, and multiplexers to control operation of the sensors. A storage memory is connected to the microcontroller to accommodate program instructions as well as data storage. Power can be provided, e.g., by a battery or by a power supply. The apparatus can communicate with a user or with other systems via a display, a touch screen or buttons, and wireless communication facility.

In operation, measurements can be made repeatedly or upon initiation, e.g. by a user button press or wireless signal. The microcontroller configures the dual switch and the multiplexers.

It is believed that the blood pressure affects the density of the tissue, and the relative proportions of constituents of the tissue, and that those changes result in changes in electrical properties that enable the present measurement. Diastolic pressure in particular is believed to be related to the tissue density and constituents because it is influenced by blood flow restrictions associated with high blood pressure. The foregoing is the inventors' current understanding, and should not be construed as limiting the invention to any particular theory of operation.

### Example Device Embodiments

In accordance with an example embodiment, a device uses skin capacitance, resistance, and phase to enable a determination of blood pressure.

A measurement of the surface capacitance (where capacitance means the resistance to flow of alternating current), resistance (where resistance means the resistance to flow of direct current) and phase (the relationship between capacitance and resistance) can be made with various suitable meters; however, the meters must be coordinated to enable detection of the desired capacitance, resistance and phase. The “surface capacitance” of a subject is the capacitance of the top layer of the subject's epidermis and can be measured by capacitance as measured by higher frequencies and closer spaced contacts. The “surface resistance” of a subject is the resistance of the top layer of the subject's epidermis can be measured by DC voltage and closed spaced contacts. The “surface phase” of a subject is the relationship between the two above, e.g., the relative magnitudes of the two.

The sub surface capacitance of a subject is the capacitance of the internal layer of the subject's dermis and can be measured by capacitance as measured by lower frequencies and larger spaced contacts. The “sub surface resistance” of a subject is the resistance of the internal layer of the subject's dermis and can be measured by lower frequencies and larger spaced contacts. The “sub surface phase” of a subject is the relationship between the two above, e.g., the relative magnitudes of the two.

FIG. 1 illustrates an embodiment of a circuit diagram of the capacitance meter of the present invention. FIG. 2 is a schematic illustration of an interdigitated sensor suitable for use with the present invention. The example comprises three separate conductive entities, designated in the figure as CONTACT A, CONTACT B, AND CONTACT C. These three contacts can be utilized in various combinations, in pairs or all three at the same time, to effectively measure different locations and depths in the tissue. Other embodiments can have more or fewer contacts, and various patterns of interdigitation are contemplated other than the simple comb-like structure shown. As examples, parallel spirals, concentric part circles, two dimensional and three-dimensional arrays of contacts, fractal structures provide a large number of small, spatially related contacts, contacts with different width electrical conductors (that can correlate with different power injection characteristics), can be suitable.

The general skin capacitance of individuals can vary. Varying the spatial frequency of the interdigitated contact area changes the capacitance range and allows the device to be selectively matched to users with different baseline capacitances resistance and phases. In an example embodiment, the spatial frequency of the interdigitated area is approximately 1, 2, and 3 per mm. In an example embodiment, the spatial frequency of the interdigitated area is equal or greater than 1 per mm.

The contact area can also be interdigitated, electrically separated, stainless (or other non-corroding, coated conductor) wires. The interdigitated contact area can be made of stainless steel or individual wires coated to prevent corrosion, or an exposed coated printed circuit board or foil. In an example embodiment the contact area can be a single use contact surface. This can reduce eliminate complications that may arise due to surface cleaning or degradation of the contact area.

A power supply section of the meter can be of any suitable type and is illustrated in an alternating current (AC) signal, or equivalent. In an example embodiment the supplied voltage is a variable alternating current ranging from DC or zero or a few hertz to the high M Hz range. This concentrates the current flow on the surface of the skin to further differentiate skin capacitance from subcutaneous capacitance. High frequencies can be useful to concentrate the current flow on the surface of the skin.

In an example embodiment of the present invention, the device includes both low frequency and high frequency AC. In this embodiment the current flow difference measured across the contact area using both low frequency and high frequency AC is compared. This contrasts the contributions to capacitance that comes from the subcutaneous path to that of the surface capacitance, and can be used to determine depths in the tissue that correlate well in changes due to changes in blood pressure.

A device of the present invention, in an example embodiment, includes a processing system and it can also include a memory module and a transmitter to communicate with a smart phone for display of a blood pressure indication, as discussed herein.

The processing system can be a programmable processor included to execute program instructions to guide a data process module to directly store captured data into the memory module, to initiate data upload from the memory module to a computing means via data upload module, to compare current readings to initial reading, to manage energy usage and so forth.

The memory module can include any appropriate type of memory now known or later developed including without limitation, read-only memory (ROM), random access memory (RAM), flash memory, and a set of registers included within the programmable processor.

In an example embodiment, the processing system can comprise a microprocessor such as the Maxim Health Sensor Platform MASXREFDES100#. Maxim MAXREFDES100# health sensor platform is an integrated sensor platform that helps customers evaluate Maxim's complex and innovative medical and high-end fitness solutions. The platform integrates one biopotential analog front-end solution (MAX30003), one pulse oximeter and heart-rate sensor (MAX30101), two human body temperature sensors (MAX30205), one 3-axis accelerometer, one 3D accelerometer and 3D gyroscope, and one absolute barometric pressure sensor. As will be apparent to one of skill in the art, in other embodiments, other suitable processing means can also be used. In an example embodiment, blood pressure is determined from the absolute value of capacitance, and the relative magnitudes of phase and resistance.

In another embodiment, the device of the present invention can include wireless communication capabilities that would allow it to connect with a computing device, such as a laptop, a desktop, wireless devices such as cellular phones and pads, or to directly connect to the Internet. The information stored in the memory means can be downloaded into a computing device or a cloud system for storage or for further analysis or displaying results of measurement. Specifically, in some embodiments, the device is arranged to pass on relevant information or data or share relevant information or data with any interested party or device. For example, relevant information or data can be passed to a storage device and/or a computing device such as for example but by no means limited to a server, cloud, tablet, PC, smartphone or other device for further data processing, post-processing data analysis and/or display or tabulation of results. For example, the data from many users can be shared and analyzed, for example, anonymously, at a single data storage site or storage entity.

In other embodiments, the device can comprise a microcontroller, memory, a display for visualization of data, a touch screen for user input, a battery and at least one sensor.

A device of the present invention may be used on any part of the body. The top of the fingertip is suited for measuring skin resistance, capacitance, and phase. The inside of the forearm is another suitable site. The ear lobe is another suitable site. The underside of the wrist is another suitable site.

### Example Methods

Skin capacitance resistance and phase measurement are compared to a baseline values taken when the subject is measuring their blood pressure using a different device such as a blood pressure cuff, that is, the baseline skin capacitance resistance and phase values can represent the “normal” skin capacitance measurement or value of the subject.

Alternatively, in other embodiments, blood pressure can be determined directly by referencing the skin capacitance, resistance and phase measurements values determined from testing on a variety of people. The capacitance, resistance and phase measurements can be correlated with reference blood pressure measurements, and the correlation stored as a table, or analyzed to produce a mathematical relationship, using calibration techniques known in the art. The calibration can be made using measurements from a single user (a user-specific calibration), or can be made using measurements from a plurality of users (a generic calibration). A generic calibration can be generic to all users, or can be limited to users within a single physiological category, e.g., weight, body mass index, body composition, age, ethnicity, etc. A calibration can also use such physiological information as part of the calibration, and can also use other information such as temperature, tissue hydration, etc. to facilitate accurate blood pressure reports.

The inventors have observed that tissue capacitance and tissue resistance are exponentially and inversely related. Tissue resistance decreases as tissue density decreases, both tissue resistance and capacitance show little change until tissue density reaches a threshold value after which tissue capacitance increases dramatically; however, these occur at opposite ends of their respective ranges. Sensor systems that allow selection of contact spacing and frequencies, like those described herein, can allow for successful sensing even when measurements would be off-the-scale for one spacing between contacts, or essentially unchanging for another spacing between contacts.

For small spaces (e.g. 0.001 to 0.003 in) between the interdigitated strips of the contact sensor, the capacitance is high and resistance is low. For larger spaces the inverse is true: capacitance is low and resistance is high. Due to the exponential nature of these two response curves it can be advantageous to use multiple spaces to determine where the best resistance and capacitance values can to achieved and compared. There can be a space that gives reasonable values for both, but this is not always adequate to determine blood pressure as we need to use that portion of the curve where the responses are substantial. The spaces should also correspond to the electrical activity in the related subdermal layers of the skin. The depth of penetration into the tissue is a function of both frequency and contact spacing (e.g., low frequencies 1 KHz to 10 KHz and large spaces 0.005 to 0.020 in) probing deeper (in the dermis and hypodermis) and high frequencies and small spaces (e.g., 25 to 100 KHz and 0.003 to 0.005 in.) reflecting the electrical activities at shallower depths (in the epidermis). It is thought that the larger spaces reflect the electrical activity at a greater depth (we measure these depths to be between 0.02 in. for a space of 0.003 in and 0.06 for a space of 0.015 inches) due to the larger path length and influence of the arc of the current as it flows through the tissue. Depth can also be differentiated by frequencies between 5 KHz and 95 KHz at any one spacing. Once the broad range for the best depth has been determined by space size, differentiation by frequency yields a more specific signal detail and can refine the depth measurements within the depth range established by the space.

At certain spaces capacitance goes down and resistance goes up as blood pressure increases whereas at other spaces it goes up. Initial searching is helpful for determining the skin thickness and the best response ranges for measure.

Blood pressure influences the different sub strata (epidermis, dermis, subcutis) of the skin differently and this might account for the inversion of capacitance and resistance measure with space/depth. The interdigitated contacts of different spaces allow us to find the optimal depth to target changes that are specific to blood pressure, which can be dependent on measurement site (e.g., forearm, wrist, finger, etc.) and can vary dependent on the user (e.g., individual variations, age, sex, body composition, ethnicity, etc.).

The appropriate depth and the frequency can be selected to give the best detail of the signals at the selected frequency, absolute values of capacitance, resistance and phase all vary with blood pressure but to varying degrees so being able to have several different spaces and multiple different frequencies allows us to find a desired operating region, e.g., with highest signal to noise ratio.

The changes in capacitance resistance and phase can be correlated to a standard cuff measure and the changes can then be given as proportional increases or decreases from the base cuff measurement. When a sufficient number of people have been measured, then capacitance, resistance and phase can be correlated directly based on the data. The details of such a calibration can depend on the specific sensor design and implementation.

Blood pressure can be measured in two ways with the capacitance resistance and phase:

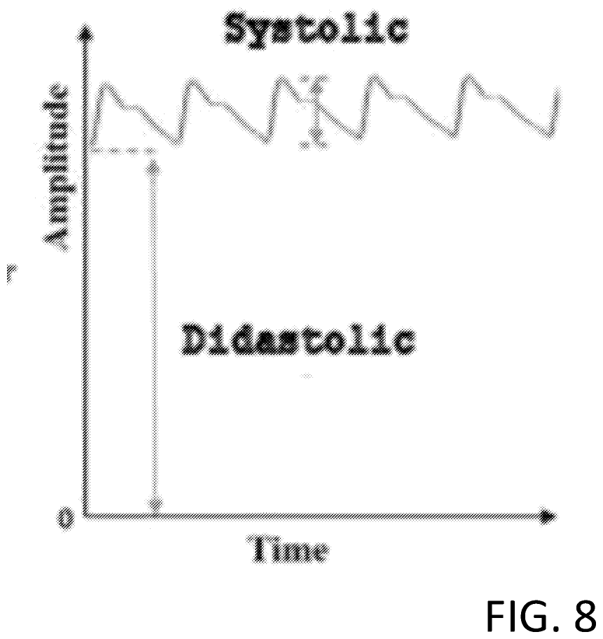

Measuring the difference between the peak capacitance or resistance or phase and the lowest capacitance or resistance or phase, allow the measurements of blood pressure directly. This is analogous to measuring the peak systolic and stable diastolic pressures in an artery, as illustrated in FIG. 8. As the tissue density changes with pulses in the blood pressure there is a rise and a fall of the electrical signal and the difference between the two can be proportional to the difference between systolic and diastolic pressures. The pulse of the subject can also affect the measurements, and the pulse can be used in combination with resistance, capacitance, and phase to further enhance performance of the system.

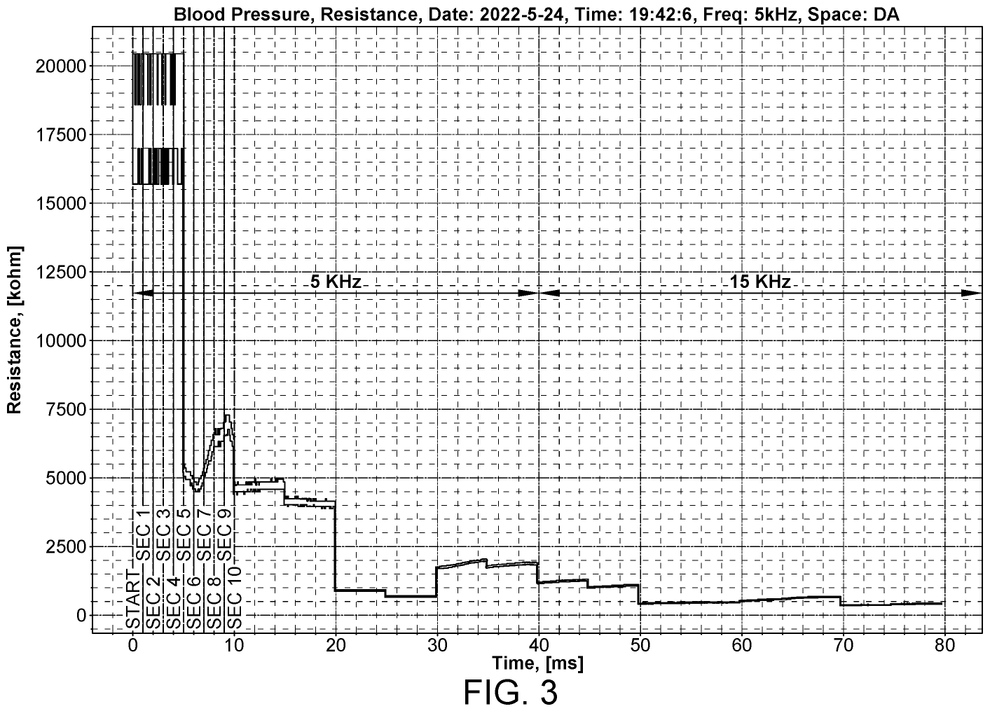

The blood pressure is proportional to the absolute value of the capacitance, resistance and phase. FIG. 3 is an illustration of blood pressure and resistance. Resistance as measured by an example embodiment is on the vertical axis. The horizontal axis is time. The illustration is separated into a plurality of periods, each of 10 seconds duration. In each period, the first 5 seconds are measured at low blood pressure; the second 5 seconds are measured at high blood pressure. Each period corresponds to a single contact spacing or measurement frequency.

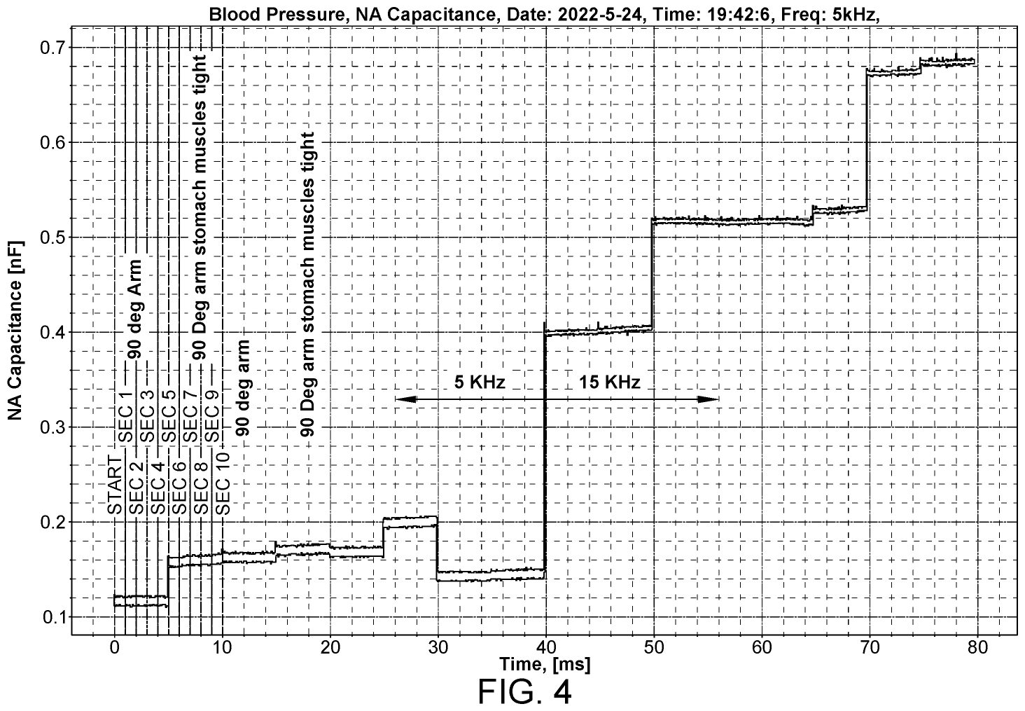

FIG. 4 is an illustration of blood pressure and capacitance. Capacitance as measured by an example embodiment is on the vertical axis. The horizontal axis is time. The illustration is separated into a plurality of periods, each of 10 seconds duration. In each period, the first 5 seconds are measured at low blood pressure; the second 5 seconds are measured at high blood pressure. Each period corresponds to a single contact spacing or measurement frequency.

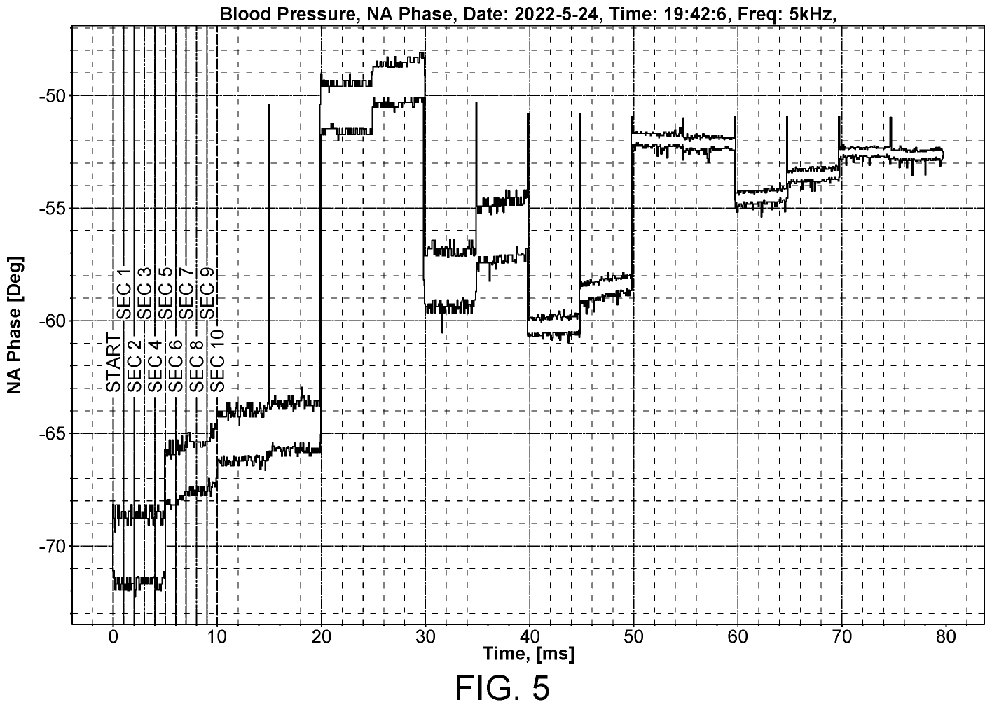

FIG. 5 is an illustration of blood pressure and phase. Phase as measured by an example embodiment is on the vertical axis. The horizontal axis is time. The illustration is separated into a plurality of periods, each of 10 seconds duration. In each period, the first 5 seconds are measured at low blood pressure; the second 5 seconds are measured at high blood pressure. Each period corresponds to a single contact spacing or measurement frequency.

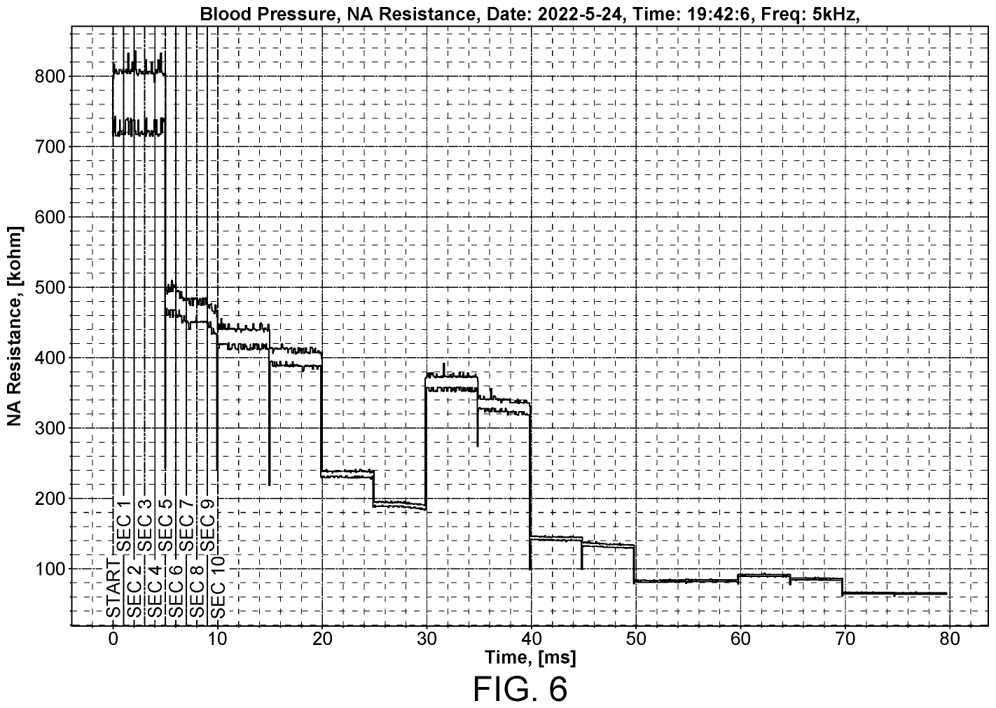

FIG. 6 is an illustration of blood pressure and resistance. Resistance as measured by an example embodiment is on the vertical axis. The horizontal axis is time. The illustration is separated into a plurality of periods, each of 10 seconds duration. In each period, the first 5 seconds are measured at low blood pressure; the second 5 seconds are measured at high blood pressure. Each period corresponds to a single contact spacing or measurement frequency.

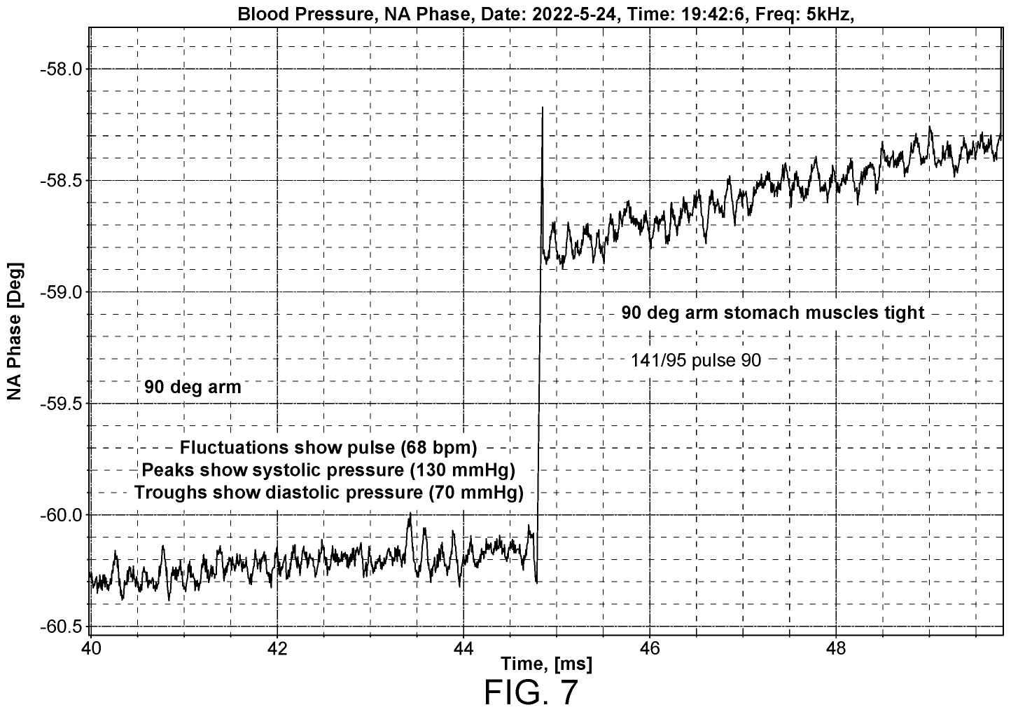

FIG. 7 is an illustration of blood pressure and phase. Phase as measured by an example embodiment is on the vertical axis. The horizontal axis is time. The illustration shows phase measurements over two 5 second periods; the first at low blood pressure and the second at high blood pressure. Fluctuations in the blood pressure measurement reflecting the pulse of the subject are visible. In each period, the first 5 seconds are measured at low blood pressure; the second 5 seconds are measured at high blood pressure. Each period corresponds to a single contact spacing or measurement frequency.

The sensor can be in a watch strap made where the longitudinal threads of the strap form the interdigitated differently spaced contact traces where one of the neighboring threads come from one edge of the watch and the other threads come from the other edge of the watch.

Blood pressure is also related to PTT (pulse transit time) which is the time between the arrival of the rise in blood pressure at two different sites. In our case the two sites are two of the different interdigitated contacts since they are spatially displaced down the arm of the user.

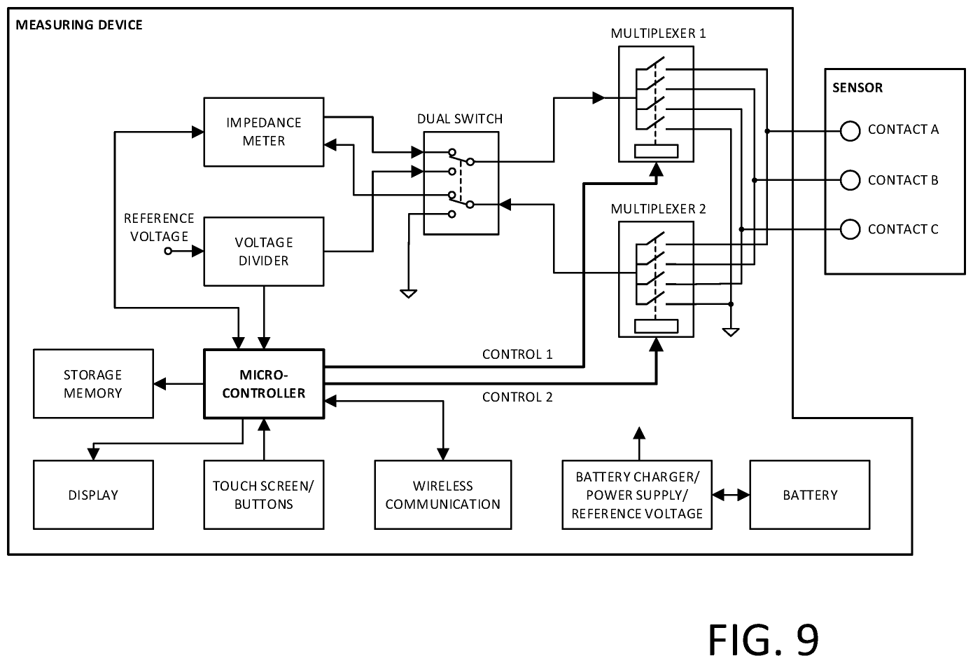

FIG. 9 is a schematic illustration of an example embodiment showing the measuring device and the sensor as separate entities.

The device can connect to and collect data from other wireless sensors located on user's body using its wireless communication module. These wireless sensors can collect the other type of data which may not be directly measured by the device, such as blood oxygen, blood glucose, body temperature, etc. Variety of data obtained by the device by direct measurement and collected from wireless sensors can be post-processed by the device itself or sent to a remote computer or a cloud system.

It is believed that one of ordinary skill in the art can, based on the description presented herein, utilize the present invention to the full extent. All publications cited are incorporated by reference.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those skilled in the art.

1. An apparatus comprising:
(i) an interdigitated portion having two opposing ends and configured for contacting skin of a subject and measuring skin capacitance, resistance and phase meter;
(ii) an analysis system that produces a signal responsive to the skin capacitance, resistance and phase measurements, where the signal is indicative of blood pressure of the subject.

2. The apparatus of claim 1 wherein the comparative capacitance resistance and phase values form a baseline capacitance resistance and phase measurement corresponding to skin capacitance resistance and phase wherein the signal indicates that the capacitance resistance and phase measurements are different from the baseline capacitance resistance and phase measurements in a repetitive manner consistent with a pulse.

3. The apparatus of claim 1, wherein the analysis system compares the skin capacitance, resistance and phase measurements to calibration skin capacitance, resistance and phase measurements and corresponding blood pressure.

4. The apparatus of claim 2, wherein the calibration measurements comprise a plurality of measurements of the present subject.

5. The apparatus of claim 2, wherein the calibration measurements comprise a plurality of measurements of a plurality of subjects.

6. The apparatus of claim 1, wherein the analysis system is further responsive to one or more of: the subject's age, weight, body mass index, ethnicity, health status.

7. A method of determining an indication of blood pressure in a subject, comprising:
(a) providing a device as in claim 1;
(b) using the interdigitated sensor at a plurality of interdigit separations to measure resistance, capacitance, and phase of a portion of the skin of the subject;
(c) comparing the measured resistance, capacitance, and phase with calibration resistance, capacitance, and phase values to determine an indication of blood pressure.

8. The method of claim 7, wherein the indication comprises an indication that the blood pressure has changed relative to the blood pressure when the calibration values were measured.

9. The method of claim 7, wherein the indication comprises an indication of the current blood pressure of the subject.

10. The apparatus of claim 2, wherein a repetitive manner consistent with a pulse means having a rhythm within range of frequencies consistent with human pulse rates.
